(19) United States
(12) Patent Application Publication
(10) Pub. No.: US 2021/0069076 A1
(43) Pub. Date: Mar. 11, 2021

(54) LUBRICATING STRIP FOR A SHAVING DEVICE, AND METHODS OF USE THEREOF

(71) Applicant: Rohan Malhotra, London (GB)

(72) Inventor: Rohan Malhotra, London (GB)

(21) Appl. No.: 16/739,738

(22) Filed: Jan. 10, 2020

(30) Foreign Application Priority Data
Sep. 5, 2019 (GB) 1912773.7

(51) Int. Cl. A61K 8/34; A61K 8/9789; B26B 21/44; A61K 8/92; A61K 8/29; A61P 17/10; A61K 8/81; A61K 47/22; A61Q 19/00; A61K 8/67; A61K 31/05; A61K 47/02; A61K 47/32; A61K 47/46; A61K 8/9794; A61K 8/25; B26B 21/52; B26B 21/22

(52) U.S. Cl. CPC A61K 8/345; B26B 21/222; B26B 21/443; A61K 8/922; A61K 8/29; A61P 17/10; A61K 8/8111; A61K 8/8117; A61K 47/22; A61Q 19/002; A61K 8/678; A61K 31/05; A61K 47/02; A61K 47/32; A61K 47/46; A61K 8/9794; A61K 8/25; B26B 21/521; A61K 8/9789

(57) ABSTRACT

A water-soluble polymer composition, which provides a slow release of an active cannabis ingredient when exposed to water, a lubricating strip comprising the water-soluble polymer, a shaving device comprising one or more of the lubricating strips, and medical uses of the same. In particular, the disclosure provides a water-soluble polymer composition which provides slow release of an active cannabis ingredient capable of being used to treat various skin conditions.

### This application claims priority to Great Britain application 1912773.7, filed Sep. 5, 2019, the entire disclosure of which is incorporated herein by reference.

### FIELD OF THE INVENTION

This disclosure relates to a water-soluble polymer composition, which provides a slow release of an active cannabis ingredient when exposed to water, a lubricating strip comprising the water-soluble polymer, a shaving device comprising one or more of the lubricating strips, and medical uses of the same. In particular, the disclosure provides a water-soluble polymer composition which provides slow release of an active cannabis ingredient capable of being used to treat various skin conditions.

### BACKGROUND

It is well known that active cannabis extracts, including cannabidiol (CBD), cannabis oil, tetrahydrocannabinol, hemp seed oil and cannabis sativa seed oil, have therapeutic uses and beneficial medicinal effects. Specifically, the dermatological benefits of applying cannabis oil, including the active ingredient CBD, to the skin are known in the art.

WO 2017/055846 A1 specifically discloses that one or more cannabinoids (including CBD) can be used in the treatment of inflammatory skin diseases such as acne, alopecia areata, basal cell carcinoma, Bowen's disease, congenital erythropoietic porphyria, contact dermatitis, Darier's disease, dystrophic epidermolysis bullosa, and eczema, including atopic eczema.

AT 0167809A also discloses the use of CBD in treating other dermatological conditions, including skin allergies and dandruff formation.

It is also widely known to incorporate lubricating strips onto the razor heads of shaving devices, which allows the blade to glide over the skin more easily during the process of shaving, and provides an overall smoother shaving experience.

EP 184440 discloses a shaving aid member including a water-leachable shaving aid composition affixed to the surface of a shaving device adjacent to the blade, which, when exposed to water, exudes a lubricating film which enhances shaving effectiveness of the razor. EP 184440 also lists polyethylene oxide (polyox) as an effective water-soluble polymer for use in the lubricating strip.

The use of therapeutic agents in the lubricating strips of shaving razors is also known in the prior art. US 20140090254 A1 relates to a lubrication strip containing thermally resilient sensate, which acts to cool the skin during shaving. US 20180133139 A1 relates to a lubrication strip containing a lubricant and a fragrance. U.S. Pat. No. 5,692,302 discloses therapeutic wound healing compositions useful for preventing and reducing injury to mammalian cells, affixed to razor cartridges to form therapeutic razor cartridges with wound healing compositions.

While the use of cannabis extracts, including CBD, to treat skin conditions in the form of a cream applied to the skin is well known, these creams often provide an instant dose of the cannabis extract. Furthermore, the cream often sits on the skin surface with much of the active cannabis extract not being efficiently absorbed into the skin.

### SUMMARY OF THE INVENTION

Accordingly, it is desirable to provide a composition that can provide an active cannabis extract to the skin to treat and/or prevent various skin conditions, and to improve the efficiency of CBD oil absorption into the skin, and a means for delivering the composition to the skin, which tackles at least some of the problems associated with the prior art. Alternatively it is desired to at least provide a commercially viable alternative to the prior art.

According to a first aspect there is provided a water-soluble polymer composition comprising from 0.1 to 5 wt. % in total of one or more ingredients selected from the group consisting of cannabidiol, cannabis oil, hemp seed oil and cannabis sativa seed oil.

### BRIEF DESCRIPTION OF THE DRAWINGS

FIG. 1 shows a shaving razor incorporating the lubricant strip disclosed herein.

FIG. 2 shows a close-up schematic of a lubricant strip as disclosed herein, provided in the head of a shaving razor.

FIG. 3 shows a schematic of the configuration of the different components in the head of a shaving device disclosed herein.

FIG. 4 shows a bar chart depicting the average scores for the five properties rated in Experimental Trial 1 (Inventive Example 1 and Comparative Example 1).

FIG. 5 shows a bar chart depicting the average scores for the three properties rated in Experimental Trial 2 (Inventive Example 2 and Comparative Example 2).

### DETAILED DESCRIPTION OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The first aspect provides a water-soluble polymer composition. That is, the composition comprises a polymer which is substantially soluble in water under shaving conditions. That is, the polymer should be soluble in water at a temperature of from 1 to 50° C., preferably from 10 to 30° C. The polymer therefore preferably has a solubility of at least 0.1 g/100 mL of water at room temperature, such as at least 1 g/100 ml and more preferably from 2 to 20 g/100 ml. Solubility can be readily measured by a person skilled in the art. The water-soluble polymer forms a majority (i.e. at least 50 wt. %, preferably at least 60wt %) of the composition.

The composition also comprises from 0.1 to 5 wt. % in total of one or more ingredients selected from the group consisting of cannabidiol, cannabis oil, hemp seed oil and cannabis sativa seed oil. The one or more ingredients of the water-soluble polymer composition described herein is preferably cannabidiol.

The water-soluble polymer composition described herein preferably comprises polyethylene oxide as the water-soluble polymer. Other polymers, including those having a lower water solubility, or which are water-insoluble, may also be included in the polymer composition, including alconox and general purpose polystyrene (GPPS). Polyox™ WSR 750 is a preferred water soluble polymer.

The water-soluble polymer composition described herein preferably comprises as the water soluble polymer a polymer with a molecular weight of approximately 300,000 Da, and a viscosity of from 600 to 1,200 cP in a 5% aqueous solution. Viscosity can be readily measured by a person skilled in the art using standard test methods, such as by the procedure described in ASTM D445, which is a capillary tube viscometer test method.

When water contacts the water-soluble polymer, it is able to form a solution with a viscosity of between 600 and 1,200 cP in 5% aqueous solution, which has excellent lubricating properties. The lubricating solution formed when water contacts the polymer composition assists the razor head containing a lubricating strip comprising the polymer composition in gliding over the skin. However, when water mixes with the polymer composition and forms a lubricant solution with a viscosity of 600 to 1,200 cP in 5% aqueous solution on the razor head, it is not viscous enough to cause any clogging of the blades.

Furthermore, a lubricating polymer solution with a viscosity in the range of 600 to 1,200 cP in a 5% aqueous solution is able to carry the one or more cannabis-based ingredients to the skin, and allows the cannabis ingredient to be easily absorbed into the skin. On the other hand, using a cream with a greater viscosity is less efficient at carrying cannabis-based ingredients to the skin, and actually inhibits the absorption of the cannabis ingredient from the cream into the skin.

The molecular weight of the polymer also impacts the viscosity of the polymer solution. A high molecular weight increases the viscosity of the material, as the longer chains make it more difficult for them to flow through the solution. A water-soluble polymer with a molecular weight of approximately 300,000 Da allows the polymer to form a polymer solution upon contact with water, having a viscosity in the preferred range of 600 to 1,200 cP in 5% aqueous solution, which displays useful properties as a lubricant in shaving applications, as described above.

The water-soluble polymer composition described herein preferably contains the one or more ingredients in an amount of from 0.2 to 3 wt. %, more preferably from 0.4 to 0.8 wt. %, even more preferably from 0.5 to 0.7 wt. %, and even more preferably about 0.6 wt. %, in total. The ingredients being provided in this amount allows the water-soluble polymer to provide the skin with the one or more cannabis-based ingredients in a medicinally effective amount, whilst also keeping the amount of ingredient sufficiently low so as to minimise the economic expense. Furthermore, providing the water-soluble polymer composition with a cannabis-based material in an amount of not greater than 5 wt. %, or in the preferred amount of 0.2 to 3 wt. %, prevents consumers from using the cannabis-based material in an abusive manner, or for recreational purposes, as this amount is not sufficient to induce any significant psychological or physiological effects upon oral consumption.

As discussed above, the water-soluble polymer composition described herein preferably comprises the one or more water soluble polymers in an amount of from 50 to 95 wt. %, more preferably from 60 to 90 wt. %, even more preferably from 70 to 85 wt. %, and most preferably in an amount of about 80 wt. %, of the total composition.

The water-soluble polymer composition described herein preferably further comprises one or more additives selected from the group consisting of vitamins, minerals, fatty acids, silica, titanium dioxide, 4,4′-Bis((4-anilino-6-((2-hydroxyethyl)methylamino)-s-triazin-2-yl)amino)2,2′-stllbenedisulfonate (Tinopal), Aloe Vera, Tea Tree Oil, grapeseed oil, non-water-soluble polymers, and butter, including cocoa butter and shea butter. Silica additives which may be suitable for use in the composition described herein include synthetic amorphous pyrogenic silica, such as Cabosil. These can provide improved properties, including colour, fragrancy and consistency to the composition.

According to another aspect, there is provided a lubricating strip comprising the water-soluble polymer described herein, for use with a shaving device.

The lubricating strip described herein preferably has a weight of from 0.1 to 2 g, or more preferably of from 0.2 to 1 g. The lubricating strip may also have a thickness orthogonal to the plane of the strip of no greater than 3 mm, or preferably of no greater than 2.5 mm, or more preferably no greater than 2 mm. This thickness of the lubricating strip allows it to be easily affixed to the razor head, either by injection moulding or by casting. The more material in the strip the larger the amount of active which can be present, but there are constraints on the space available on the razor head and the variance of strip thickness that still permits the blades to be effectively used.

The thickness of the lubricating strip is also preferably at least 0.2 mm, or more preferably at least 0.3 mm, or even more preferably at least 0.5 mm. Providing a lubricating strip with a thickness at least 0.2 mm means that the strip is capable of providing a meaningful amount of the one or more cannabis ingredients for multiple individual uses of the razor containing the lubricating strip. The razor head can therefore be used for a significant period of time, before needing to be replaced with a new razor head containing a fresh lubricating strip.

The specific preferred thickness of 0.2 mm to 3 mm, or more preferably of 0.3 mm to 2 mm, allows the razor head to be used a number of times, so that the lubricating strip would be used up at approximately the same time as the blades of the razor would begin to become blunt, therefore requiring the razor head to be replaced. This specific thickness of the lubricating strip means that neither the blades or the lubricating strip need to be replaced much earlier than the other (i.e. both the blades and lubricating strip have a similar use lifetime).

According to another aspect, there is provided a shaving device comprising one or more lubricating strips as described herein.

The shaving device described herein may comprise a razor head, wherein the one or more lubricating strips is affixed to the razor head of the shaving device. This close proximity of the lubricating strip to the razor blades on the razor head allows the lubricating strip to provide a smoother shave. Furthermore, the gentle compression of the razor head on the skin stimulates the skin to absorb more of the one or more active cannabis ingredients from the polymer lubricating solution that is spread on the skin during shaving.

The lubricating strips are preferably affixed to the razor head of the shaving device described herein by injection moulding, or by casting. The one or more lubricating strips may also be positioned next to one or more of the blades of the razor head, such that the lubricating strip is configured to contact the skin immediately preceding, during or following passage of the blade on the skin. The close proximity of the one or more lubricating strips to the blades of the razor head increases the efficiency of the absorption of the one or more cannabis ingredients by the skin. The gentle compression of the razor head on the skin stimulates the skin to absorb more of the one or more active cannabis ingredients from the polymer lubricating solution that is spread on the skin during shaving.

According to a further aspect, there is provided a polymer composition as described herein, for use in the treatment or prevention of a skin condition, wherein the polymer composition is provided as a lubricating strip as described herein, or is provided in a shaving device as described herein.

According to a further aspect, there is provided a polymer composition as described herein, for use in a method for the treatment or prevention of a skin condition, wherein the method comprises contacting the skin to be treated with a shaving device as described herein.

According to a further aspect, there is provided a polymer composition as described herein, for use in a method for the treatment or prevention of a skin condition, wherein the method comprises contacting the skin to be treated with a shaving device comprising the polymer composition, wherein the polymer composition is affixed to the razor head of the shaving device.

The skin is preferably contacted whilst shaving hair from the adjacent skin surface of the shaving device with the polymer composition for use as described herein.

The skin condition may be one or more of the conditions selected from the group consisting of acne, excessively oily skin, excessive sebum production, insect bites, infections, wounds, inflammation, eczema, burns, including razor burns, ingrown hairs, dry skin, skin wrinkles and clogged pores. The use of the polymer composition in a lubricating strip of a shaving device is particularly advantageous in treating conditions which are aggravated by or caused by shaving. The polymer composition containing the one or more active cannabis ingredients is able to effectively treat conditions such as eczema, burns, ingrown hairs, wounds, acne, and dry skin, which are all susceptible to irritation during the shaving process.

Furthermore, when the shaving device containing the lubricant strip described herein is used on a regular basis, the skin conditions disclosed herein can be effectively treated, and the symptoms reduced over time. The shaving device described herein can therefore be used for effective long-term treatment plans, in place of other treatment options such as oral medication, which may have undesirable side effects. For example, oral treatments for acne can have various side effects and health risks, which are avoided by instead using a shaving device comprising the lubricating strip described herein, on a regular basis, such as on a daily basis.

The polymer composition for use as described herein is preferably the lubricating strip as described herein.

The present inventors sought to provide a means for providing a meaningful amount of an active cannabis extract to the skin. Surprisingly, they found that using a water- soluble polymer composition comprising 0.1 to 5 wt. % of a compound selected from the group consisting of cannabidiol, cannabis oil, hemp seed oil and cannabis sativa seed oil, in a lubricating strip for a shaving device is able to provide a slow release of an effective amount of the therapeutic cannabis extract to the skin. In particular, the Inventors surprisingly discovered that cannabidiol (CBD) can be provided in a water-soluble polymer composition, and that this can be used to deliver a meaningful amount of active CBD ingredient to the skin overtime, which is effective at improving and/or preventing common skin conditions, such as acne, razor burns, ingrown hairs and eczema. Moreover, because the use of the razor is part of daily routine, it can be ensured that an effective dose can be provided as part of a CBD delivery treatment regime.

Furthermore, the Inventors found that using the water-soluble polymer composition containing the one or more active cannabis ingredients as a lubricant strip in a shaving device is particularly effective in treating skin conditions which are aggravated by shaving, such as acne, or which are caused by shaving, such as razor burns.

The Inventors also surprisingly found that the use of water-soluble polymer lubricating strips containing the one or more cannabis ingredients on shaving devices meant that the gentle compression of the shaving device on the skin was able to stimulate efficient uptake of the slow-release cannabis extract, resulting in improved therapeutic effects when a lower dose of cannabis extract is provided, in comparison to the amount absorbed by the skin when applied as a cream.

The lubricating strip applied to a shaving device described herein has been described in the configuration of a shaving device primarily designed for and used by males. However, the polymer composition can also be applied as lubricating strips to shaving devices typically designed for and used by women, including incorporating a lubricating strip that is affixed around the entire edge of the razor head (i.e. forming a band around all edges of the razor blades) to give a more cushioned and smooth shave. Furthermore, although the beneficial effects of the polymer composition described herein have been detailed in relation to skin conditions often found on the facial region, the polymer composition described herein can also be used on shaving devices for use on other regions of the body, such as underarms, legs and intimate regions.

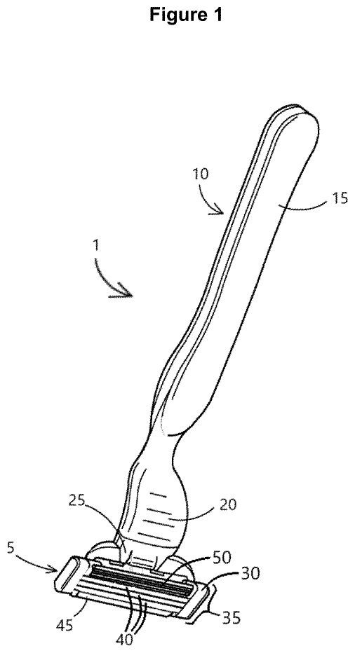

FIG. 1 shows a shaving razor (1) formed from a head (5) and a body (10). The body (10) has a handle portion (15) and an angled attachment portion (20). The angled attachment portion (20) is releasably connected to the head (5) by a click mechanism (not shown).

The head (5) is formed from a retention portion (25) and a skin contacting portion (30). Skin contacting portion (30) is pivotably mounted on the retention portion (25). The skin contacting portion (30) has a skin-contacting surface (35). The skin contacting surface (35) comprises a plurality of angled razor blades (40) and first and second lubricant strips (45, 50).

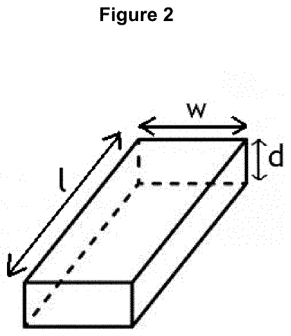

The first and second lubricant strips are each about 2.5 cm in length (l) across the skin contacting surface (35). The thickness (d) of each strip is about 1 mm. The width (w) of each strip is about 4 mm. The total weight of each strip is about 0.4 g. These relative dimensions are shown in FIG. 2.

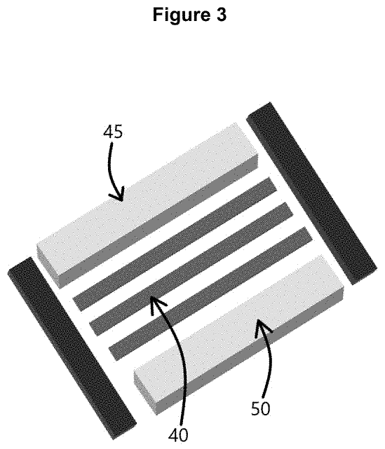

The first and second lubricant strips (45, 50) are positioned at opposite sides of the plurality of angled razor blades (40) on the skin contacting portion (30). The lubricant strips (45, 50) are positioned parallel to the razor blades (40), with the length (l) of the lubricant strips (45, 50) being of approximately the same length as the angles razor blades (40). This configuration is shown in FIG. 3.

In use, the skin contacting surface (35) is drawn across an area of skin to be shaved. The razor blades (40) are configured to pare hairs from the surface. The first lubricant strip (45) which precedes the razor blades (40) provides the skin with a lubricant film. The second lubricant strip (50) which follows the razor blades (40) provides the skin with a moisturising film. The first and/or second lubricant strip can be formed of the composition described herein.

The following composition is an embodiment of the composition described herein:

### POLYOX WSR N-750≥30%

### ALCONOX WSR≥30%

### GPPS≥20%

### TITANIUM DIOXIDE≥2%

### CABOSIL≥5%

### TINAPOL≤1-2%

### VITAMIN E≤1-2%

### ALOVERA≤1-2%

### TEA TREE OIL≤1-2%

### CBD OIL≤0.1-5.0%

Polyox™ WSRN 750 is comprised of:

Poly(ethylene oxide)≥95.0%
Fumed Silica (generic)≤3.0%
Calcium as mixed salts≤1.0%.
with wide ranging molecular weights from 100,000 to 8,000,000. POLYOX Water-soluble Resins are non-ionic, high molecular weight water-soluble poly (ethylene oxide) polymers.

The water-soluble polymer compositions described herein were affixed as lubricating strips to a razor head and tested. It was surprisingly found that the compositions had a beneficial effect in treating a range of skin conditions, such as acne, eczema, razor burns, and ingrown hairs.

The following Examples illustrate the unexpected beneficial effects of using the water-soluble polymer compositions described herein as lubricating strips affixed to a razor head.

### EXAMPLES

### Experimental Trial 1

A trial was carried out to test the effectiveness of a disposable razor with a CBD-containing lubra strip in treating/preventing (1) acne and skin breakouts, (2) post-shave skin irritation and/or razor burn, (3) ingrown hair, (4) oily skin, and (5) dry and/or rough skin. The results were analysed to generate average scores, and compared to the results of an equivalent test that was carried out using a disposable razor without a CBD-containing lubra strip.

### Inventive Example 1

Identical samples of a disposable face shaving razor with three blades and a CBD-containing lubra strip in accordance with the invention were prepared. The CBD-containing lubra strip on each razor contained 30% Polyox, 30% Alconox, 20% GPPS, 5% Full-spectrum CBD oil, 5% Cabosil, 1% Tinapol, 2% Aloe Vera, 2% Vitamin E oil, 2% titanium dioxide, 1% grapeseed oil, 1% tea tree oil, and 1% shea butter.

50 male subjects were selected to test the disposable face shaving razor with a CBD-containing lubra strip. The 50 male participants were selected due to each participant having one or more of the following complaints about their regular shaving experiences:

Post-shave irritation;

Post-shave acne;

Post-shave oily skin;

Post-shave Ingrown hair; and

Post-shave dry and rough skin.

The 50 male subjects were asked to shave their facial area every alternate day for 10 days (i.e. a total of 5 consecutive shaves with a one-day gap between each shave). These gaps were intentionally stipulated to allow a time interval for the CBD oil to take effect and subsequent post-shave effects of using the razor with a CBD oil lubra strip to be observed. Shaving of the male participants' facial region predominantly included shaving the beard and moustache regions of the face.

At the end of the 10-day trial, the 50 male subjects were then asked to rate their shaving experiences by providing a value of from 1 to 10 for the following five properties:

1) Acne and/or breakouts—this refers to the amount and severity of the acne and spot breakouts that occur on the facial region following shaving. This property was rated on a scale of from 1 to 10, were 1 means absolutely no acne or breakouts of spots on the skin of the facial region where shaving had been carried out, and a score of 10 means severe acne and spot breakouts on the skin of the facial region where shaving had been carried out.

2) Post-shave skin irritation and/or razor burn—this refers to the irritation felt on the region of the skin that has been subjected to shaving, and/or the razor burn experienced on the skin caused by shaving. This property was rated on a scale of from 1 to 10, were 1 means absolutely no skin irritation and/or razor burns on the skin of the facial region where shaving had been carried out, and a score of 10 means severe skin irritation and/or razor burns on the skin of the facial region where shaving had been carried out.

3) Ingrown hair—this refers to ingrown hairs that form after shaving. Ingrown hairs occur when the hair curls back or grows inwards or sideways into the skin. Ingrown hairs cause discomfort and may also cause a red irritable lump on the surface of the skin. This property was rated on a scale of from 1 to 10, where 1 means absolutely no ingrown hairs on the skin of the facial region where shaving had been carried out, and a score of 10 means a very large amount and severity of ingrown hairs on the skin of the facial region where shaving had been carried out.

4) Oily skin—this refers to the amount of oil observed on the skin of the facial region where shaving was carried out. This often occurs due to aggravation of the sebaceous gland during shaving. This property was rated on a scale of from 1 to 10, were 1 means absolutely no oil was observed on the skin of the facial region where shaving had been carried out, and a score of 10 means that a large amount of oil was present on the skin of the facial region where shaving had been carried out.

5) Dry and/or rough skin—this refers to the amount and severity of skin that is observed as dry and/or rough on the facial region that has been subjected to shaving. Dry skin is often observed as being flaky and/or of rough texture. This property was rated on a scale of from 1 to 10, were 1 means absolutely no dry and/or rough skin on the facial region where shaving had been carried out, and a score of 10 means severe dry and/or rough skin on the facial region where shaving had been carried out.

The results of the experiment were collected, and the scores were analysed to produce average scores, as shown in Table 1 below.

TABLE 1

| Property | Average score |
|---|---|
| 1) Acne and/or breakouts | 1.84 |
| 2) Post-shave irritation | 1.82 |
| and/or razor burns | |
| 3) Ingrown hairs | 1.82 |
| 4) Oily skin | 1.78 |
| 5) Dry and/or rough skin | 1.7 |

### Comparative Example 1

A comparative test was carried out to assess the difference in average score when an equivalent razor without a CBD-containing lubra strip was used. The resulting data was then analysed.

10 male participants were selected at random from the original 50 male participants that took part in the experiment using the razor with a CBD lubra strip. The 10 male participants were asked to shave their facial area every alternate day for 10 days (i.e. a total of 5 consecutive shaves with a one-day gap between each shave). Shaving of the male participants' facial region predominantly included shaving the beard and moustache regions of the face.

At the end of the 10-day trial, the 10 male participants were asked to rate their shaving experiences using the same properties and scale used to rate the disposable razor with a CBD-containing lubra strip.

The results of the comparative experiment were collected, and the scores were analysed to produce average scores, as shown in Table 2 below.

TABLE 2

| Property | Average score |
|---|---|
| 1) Acne and/or breakouts | 2.9 |
| 2) Post-shave irritation | 3.4 |
| and/or razor burns | |
| 3) Ingrown hairs | 2.9 |
| 4) Oily skin | 2.4 |
| 5) Dry and/or rough skin | 2.8 |

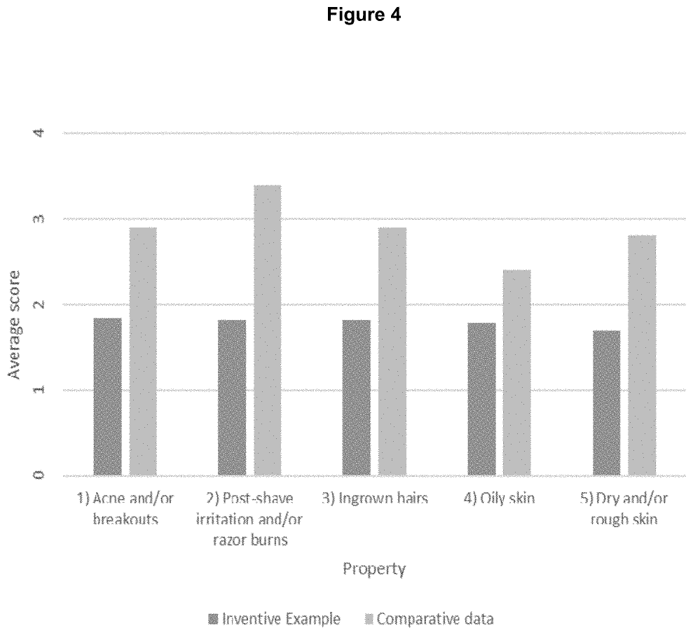

The average scores of the five properties rated for the inventive example and Comparative Example 1 are shown in the bar chart of FIG. 4.

As can be seen, the average score for all five of the properties rated were lower after using the disposable razor with the CBD-containing lubra strip (i.e. the inventive example), compared to the average scores obtained after using the disposable razor with a lubra strip that did not contain any CBD (i.e. Comparative Example 1).

The results of the experiments clearly show that the disposable razor of the inventive example demonstrates various unexpected technical effects when used regularly.

Firstly, it is clear that using the disposable razor with the CBD-containing lubra strip has positive effects in reducing the signs of acne and/or spot breakouts on the skin, which are often aggravated and worsened as a result of shaving. As the lubra strip reacts with the water to become lubricating, there is provided a slow release of the CBD onto and into the skin. This slow release of CBD onto the skin mitigates the excessive sebum production and normalises the sebaceous gland, which in turn prevents oiliness and clogged pores, resulting in a reduction in acne and spot breakouts. Furthermore, traditional shaving is known to aggravate the clogged pores and existing spots, which often worsens the spots and acne. The disposable razor with the CBD-containing lubra strip helps to resolve this problem.

Secondly, the data shows a clear reduction in the amount and severity of post-shave skin irritation and/or razor burns. Shaving is known to cause skin irritation, and can frequently result in razor burns, which generally reside as uncomfortable redness of the skin, stinging and bumps on the skin. This can be both unsightly and cause discomfort and irritation. The razors with a CBD-containing lubra strip prevent the formation of razor burns and skin irritation by acting as a deterrent. The CBD released from the lubra strip has analgesic and anti-inflammatory properties which are released onto the skin when the lubricating strip is gently compressed onto the skin.

Thirdly, a reduction in the amount and severity of ingrown hairs was observed for the inventive example compared to Comparative Example 1. Ingrown hairs are a common problem of shaving, and especially for those who have curly hair. When hairs are shaved, and especially when harsh pressured is applied to the surface of the skin, the growth journey of the hair turns inwards leading to the hair growing into and under the surface of the skin. This can lead to severe discomfort and unsightly red lumps on the skin. The gentle compression and slow release of CBD during shaving using a razor with the CBD-containing lubra strip acts to counter this problem. In particular, the CBD assists in preventing the formation of ingrown hair by reducing the harshness of the shave and further making shaving a soothing experience with less irritation.

Fourthly, the average score for the amount of oil on the participants' skin was lower for the inventive example than for Comparative Example 1. Shaving, and especially shaving of the facial region, aggravates the sebaceous gland, and stimulates the release of oil onto the surface of the skin. This both encourages the formation of spots, and is unsightly. The CBD released onto the skin when shaving using a razor with a CBD-containing lubra strip mitigates excessive sebum production and normalises the sebaceous gland. This, in turn, prevents oiliness and clogged pores.

Lastly, the data shows a reduction in the average score for dry and/or rough skin for the inventive example over Comparative Example 1. Dry and/or rough skin is one of the most common side effects of shaving with a razor, as the razor is harsh on the skin and depletes the skin of essential nutrients. The CBD in the lubra strip, when mixed with the lubricant in the strip, causes a slow release of endo-cannabinoids, which results in the release of antioxidants to combat free radicals and other oxidative stress that contributes to drying out the skin. Shaving with a razor that has a CBD-containing lubra strip helps to repair the skin, moisture and nourish the skin, which stops the skin from drying out and thus also prevents rough skin from developing. This can make the skin look younger and can assist in boosting the production of collagen in the skin. It is also thought that the slow release of CBD onto the skin stimulates the release of vitamins A, D and E, and fatty acids like omega-6 and gamma-linoleic acid (GLA), which moisture the skin and prevent it from becoming dry and rough.

### Experimental Trial 2

A further trial was carried out to test the effectiveness of a shaving razor with a CBD-containing lubra strip in treating/preventing skin breakouts (spots), skin irritation and dry skin that result from shaving. The results were analysed to generate average scores, and compared to the results of an equivalent test that was carried out using a commercial Gillette razor.

### Inventive Example 2

Ten men were selected at random and asked to shave their facial region using a razor with a CBD-containing lubra strip. The men were then asked to rate the razor on the effect that it had on the following properties.

1) Skin breakouts—this refers to the amount and severity of the spot breakouts that occur on the facial region following shaving. The razor was rated on its effectiveness at treating and/or preventing skin breakouts on the facial region where shaving had been carried out. The following scale was used:

| | |
|---|---|
| Poor | 5 |
| Fair | 10 |
| Good | 15 |
| Very good | 20 |
| Exceptional | 25 |

2) Skin irritation—this refers to the irritation felt on the region of the skin that has been subjected to shaving. The razor was rated on its effectiveness at treating and/or preventing skin irritation on the facial region where shaving had been carried out using the same scale as for property (1).

3) Skin dryness—this refers to the amount and severity of skin that is observed as being dry and on the facial region that has been subjected to shaving. Dry skin is often observed as being flaky and/or of rough texture. The razor was rated on its effectiveness at treating and/or preventing dry skin on the facial region where shaving had been carried out using the same scale as for property (1).

The results of the experiment were collected, and the scores were analysed to produce average scores, as shown in Table 3 below.

TABLE 3

| Property | Average score |
|---|---|
| 1) Skin breakouts | 22 |
| 2) Skin irritation | 24 |
| 3) Dry skin | 23.5 |

### Comparative Example 2

In this additional trial, a comparative test was carried out to assess the difference in average score when a commercially available Gillette razor was used. The same ten men that took part in the experiment of Inventive Example 2 were asked to shave their facial region using a commercial Gillette razor, and then rate the razor on the effect that it had on the same properties that were rated in Inventive Example 2 (and using the same scale).

The resulting data was then analysed to generate average scores, which are shown below in Table 4.

TABLE 4

| Property | Average score |
|---|---|
| 1) Skin breakouts | 5.5 |
| 2) Skin irritation | 10 |
| 3) Dry skin | 10 |

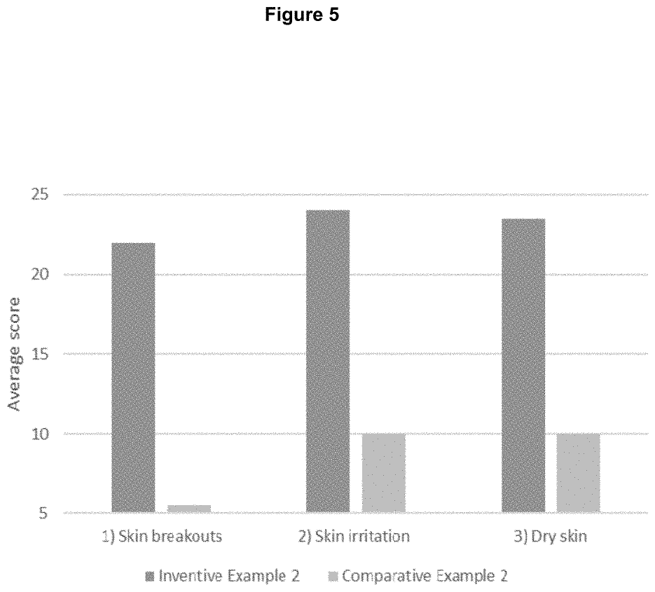

The average scores of the three properties that were rated in Experimental Trial 2 (for Inventive Example 2 and Comparative Example 2) are shown in the bar chart of FIG. 5.

As can be seen from FIG. 5, the razor of Inventive Example 2 (with the CBD-containing lubra strip) has far higher (i.e. better) average scores for all of the three properties rated (i.e. treatment/prevention of skin breakouts, skin irritation and dry skin). This significant difference in average scores demonstrates that the razor with the CBD-containing lubra strip used in the experiment of Inventive Example 2 is able to provide a slow release of CBD, which in turn has a positive effect on reducing and/or preventing breakouts of spots on the skin, as well as reducing and/or preventing skin irritation caused by shaving and reducing and/or preventing the formation of dry skin that often results from shaving the facial region.

Unless otherwise stated, all percentages herein are by weight.

Although preferred embodiments of the invention have been described herein in detail, it will be understood by those skilled in the art that variations may be made thereto without departing from the scope of the invention or of the appended claims.

1. A water-soluble polymer composition comprising from 0.1 to 5 wt. % in total of one or more ingredients selected from the group consisting of cannabidiol, cannabis oil, hemp seed oil and cannabis sativa seed oil.

2. The water-soluble polymer composition of claim 1, wherein the polymer composition comprises polyethylene oxide.

3. The water-soluble polymer composition of claim 1, wherein the polymer composition comprises one or more polymers with a molecular weight of approximately 300,000 Da, and a viscosity of from 600 to 1,200 in a 5% aqueous solution.

4. The water-soluble polymer composition of claim 1, wherein the one or more ingredients is present in an amount of from 0.2 to 3 wt. % in total.

5. The water-soluble polymer composition of claim 4, wherein the one or more ingredients is present in an amount of from 0.4 to 0.8 wt. %, preferably about 0.6 wt. % in total.

6. The water-soluble polymer composition of claim 1, wherein the polymer composition comprises one or more polymers present in an amount of from 50 to 95 wt. %, preferably from 60 to 90 wt. %, or more preferably from 70 to 85 wt. %, by weight of the total polymer composition.

7. The water-soluble polymer composition of claim 6, wherein the one or more polymers is present in an amount of about 80 wt. % of the total polymer composition.

8. The water-soluble polymer composition of claim 1, further comprising one or more additives selected from the group consisting of vitamins, minerals, fatty acids, silica, titanium dioxide, 4,4′-Bis((4-anilino-6-((2-hydroxyethyl)methylamino)-s-triazin-2-yl)amino)2,2′-stllbenedisulfonate, Aloe Vera, Tea Tree Oil, grapeseed oil, non-water-soluble polymers, and butter, including cocoa butter and shea butter.

9. A lubricating strip comprising the water-soluble polymer of claim 1, for use with a shaving device.

10. The lubricating strip of claim 9, wherein the strip has a weight of from 0.1 to 2 g and, optionally, a thickness orthogonal to the plane of the strip of no greater than 3 mm.

11. A shaving device comprising one or more lubricating strips as defined in claim 9.

12. The shaving device of claim 11, comprising a razor head, and wherein the one or more lubricating strips is affixed to the razor head of the shaving device.

13. The shaving device of claim 12, wherein the one or more lubricating strips is affixed to the razor head of the shaving device by injection moulding, or by casting.

14. The shaving device of claim 12, wherein the one or more lubricating strips is positioned next to one or more of the blades on the razor head, such that the lubricating strip is configured to contact the skin immediately preceding, during or following passage of the blade on the skin.

15. A polymer composition according to claim 1, for use in the treatment or prevention of a skin condition, wherein the polymer composition is provided as a lubricating strip.

16. A polymer composition according to claim 1, for use in a method for the treatment or prevention of a skin condition, wherein the method comprises contacting the skin to be treated with a shaving device comprising a lubricating strip comprising the polymer composition.

17. A polymer composition according to claim 1, for use in a method for the treatment or prevention of a skin condition, wherein the method comprises contacting the skin to be treated with a shaving device comprising the polymer composition, wherein the polymer composition is affixed to the razor head of the shaving device.

18. A polymer composition claim 16, wherein the skin is contacted whilst shaving hair from the adjacent skin surface with the shaving device.

19. A polymer composition of claim 15, wherein the skin condition is one or more of the conditions selected from the group consisting of acne, excessively oily skin, excessive sebum production, insect bites, infections, wounds, inflammation, eczema, burns, including razor burns, ingrown hairs, dry skin, skin wrinkles and clogged pores.

20. The polymer composition of claim 15, wherein the polymer is the lubricating strip.
